(19) United States
(12) Patent Application Publication
(10) Pub. No.: US 2015/0299000 A1
(43) Pub. Date: Oct. 22, 2015

(54) FLUID HANDLING SYSTEM

(71) Applicant: Purewater Therapeutics, LLC, Longmont, CO (US)

(72) Inventors: Mark Forrest Smith, Longmont, CO (US); W. Mark Davis, Louisville, CO (US); Justin Charles Smith, Broomfield, CO (US)

(73) Assignee: Purewater Therapeutics, LLC

(21) Appl. No.: 14/691,832

(22) Filed: Apr. 21, 2015

Related U.S. Application Data
(60) Provisional application No. 61/982,473, filed on Apr. 22, 2014.
(60) Provisional application No. 62/046,626, filed on Sep. 5, 2014.

(51) Int. Cl. C02F 1/469; C02F 1/00; A61L 2/02

(52) U.S. Cl. CPC C02F 1/4691; A61L 2/022; C02F 1/001; C02F 1/006; C02F 2103/026

(57) ABSTRACT

A fluid handling system is disclosed as it may be implemented to purify water. In an example, a single-unit fluid handling system includes a stationary filter, a mobile tank, and an end effector subsystem. The stationary filter has a Capacitive Deionization (CDI) subsystem. The stationary filter also has a mix and filter subsystem, and an output subsystem. The mobile tank has a tank subsystem, and a tank liner subsystem. The system may also include a waste handling component having a waste tank subsystem. In an example, purified fluid is maintained in physical, chemical, and biological isolation from waste fluid on the same device. Although not limited in end-use, the fluid handling system may be configured to further combine the purified water with medical fluid concentrate, store the fluid, and then dispense the product, e.g., via a medical device for use in a medical treatment procedure and/or surgery.

### CROSS REFERENCE TO RELATED APPLICATION

This application claims the priority benefit of U.S. Provisional Patent Application No. 61/982,473 filed Apr. 22, 2014 for “Water Purification System,” and U.S. Provisional Patent Application No. 621046,626 filed Sep. 5, 2014 for “Combined Water Purification System And Waste Handling System,” each hereby incorporated by reference in its entirety as though fully set forth herein.

### BACKGROUND

Sterile medical fluids are commonly distributed in bags with a volume of five liters or less. Many applications for sterile medical fluids utilize much more than the typical bag. For example, a common surgical procedure can use 30 liters or more of irrigation fluid. In the United States alone, there are currently about ten million surgical procedures performed every year that use irrigation fluid. Other applications, such as Continuous Renal Replacement Therapy (CRRT), can use more than 150 liters of fluid. Nearly 30% of ICU patients experience kidney failure, needing CRRT. There are nearly 200,000 annual CRRT treatments. In addition, there are currently over 30,000 home hemodialysis patients worldwide.

Using multiple bags per treatment presents several problems. For example, nurses have to monitor (e.g., continuously) the status of a patient's medical fluid bag(s) so that the bags do not run dry during a treatment or procedure. This distracts nurses from other duties, and over time can cause fatigue. Also for example, hospitals and ambulatory surgery centers have to continually re-order fluid and transport the fluid bags from the shipping docks, to a storage facility, and then to the point of use. By way of illustration, medical irrigation bags may include only three liters of fluid. Therefore multiple bags have to be used in most surgical cases, in hip arthroscopy, for example, as many as seven bags are routinely used. The bags are heavy (e.g., almost seven pounds), and awkward to handle because they must be hung at heights of five to six feet from the ground. In addition, the environmental impact of using all of these bags can be significant. For example, 20-30 million three liter bags are currently consumed in the US every year, which have to be transported and ultimately disposed of.

### BRIEF DESCRIPTION OF THE DRAWINGS

FIGS. 1a-c are block diagrams illustrating example fluid handling systems.

FIG. 2 illustrates an example pre-treatment subsystem of the example fluid handling system.

FIG. 3 illustrates an example Capacitive Deionization (CDI) subsystem of the example fluid handling system.

FIG. 4 illustrates an example mix and filter subsystem of the example fluid handling system.

FIG. 5 illustrates an example output subsystem of the example fluid handling system.

FIG. 6 illustrates an example filter station mechanical subsystem of the example fluid handling system.

FIG. 7 illustrates an example filter station electrical subsystem of the example fluid handling system.

FIG. 8 illustrates an example tank subsystem of the example fluid handling system.

FIG. 9 illustrates an example tank liner subsystem of the example fluid handling system.

FIG. 10 illustrates an example drug delivery subsystem of the example fluid handling system.

FIG. 11 illustrates an example mobile tank mechanical subsystem of the example fluid handling system.

FIG. 12 illustrates an example mobile tank electrical subsystem of the example fluid handling system,

FIG. 13 illustrates an example end effector subsystem of the example fluid handling system.

FIG. 14 illustrates an example waste tank subsystem of the example fluid handling system.

FIG. 15 illustrates an example waste connection subsystem of the example fluid handling system.

### DETAILED DESCRIPTION

A fluid handling system is disclosed as it may be implemented to purify water and may also handle waste fluids. In an example, influent (e.g., tap water) to the fluid handling system is passed through one or more filter (e.g., a series of filters) to remove large particles, ions, organic molecules, inorganic molecules, and biological materials. Although not limited in end-use, the water purification system may be configured to further combine the purified water with medical fluid concentrate, store the fluid, and then dispense the product, e.g., via a medical device for use in a medical treatment procedure and/or surgery. The waste fluids may be stored and/or disposed of by the system.

The Environmental Protection Agency (EPA) defines Primary Drinking Water according to the National Primary Drinking Water Regulations, as suitable for human consumption. While EPA Primary Drinking Water (often referred to as “tap” water) is suitable for human consumption, this water has not been sufficiently purified to meet the standards for medical use. The fluid handling system disclosed herein receives tap water as influent, and discharges purified water as effluent. The effluent may meet and/or exceed standards for medical use. In an example, the effluent may be used as an irrigation fluid that meets and/or exceed USP and AAMI standards.

The fluid handling system may be configured for end-use according to a variety of form factors. By way of non-limiting illustration, the fluid handling system may be configured as a surgical version, an Intensive Care Unit (ICU) version, and a boom-mounted version, to name only a few examples.

In an example, the fluid handling system can be configured to store and/or produce enough on-demand sterile fluid (e.g., about 10 liters) and maintain sterility of the sterile fluid through presentation to another medical device and/or the patient. A nurse does not have to change or refill bags, thus reducing the need for human interaction with the system (e.g., after a surgery or other medical procedure has started). In an example, the fluid handling system may produce a large batch of fluid (e.g., greater than about 10 to 60 L); and maintain sterility until ready for use. Waste fluid may similarly be treated and re-used and/or held for disposal. Thus, the fluid handling system reduces shipping (e.g., relative to bagged fluid), reducing production costs, transportation costs, and waste.

In an example, the fluid handling system can be configured with consumable components, such as filters and concentrate fluids, to dramatically reduce the number of items hospitals and ambulatory surgery centers need to have on hand in order to maintain a supply of irrigation fluids. In addition to reducing production costs, this can also help reduce the environmental impact, e.g., by reducing transportation and disposal of bagged fluid.

It is noted that as used herein, the terms “includes” and “including” mean, but is not limited to, “includes” or “including” and “includes at least” or “including at least.” The term “based on” means “based on” and “based at least in part on.” The term “logic” includes, but is not limited to, computer software and/or firmware and/or hardwired configurations. The term “software” includes logic implemented as computer readable program code and/or machine readable instructions stored on a non-transitory computer readable medium (or media) and executable by a processor and/or processing unit(s).

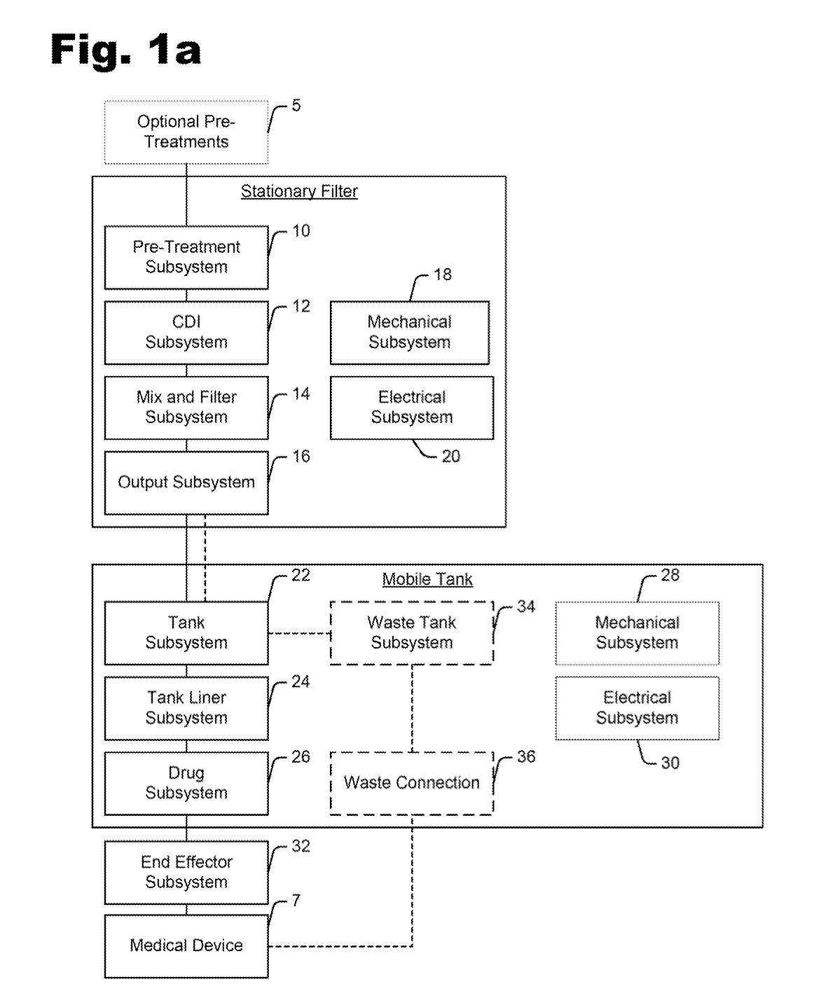

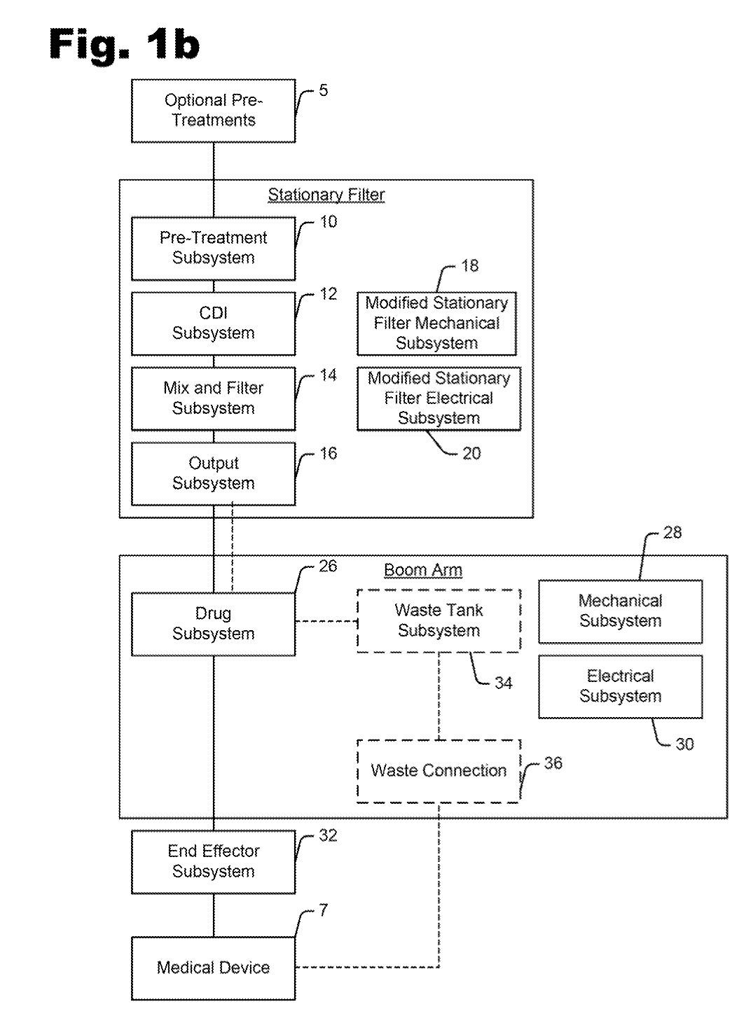

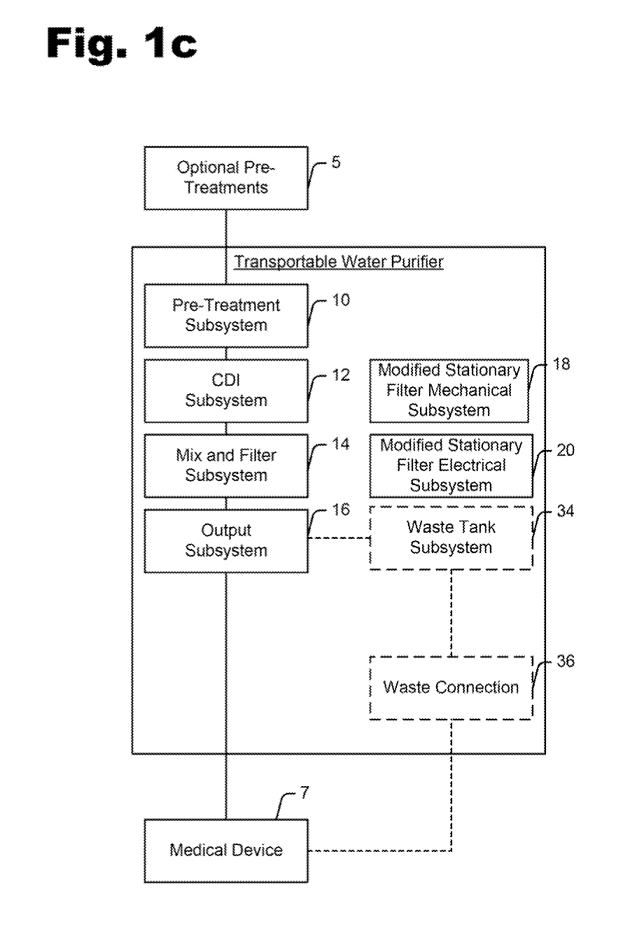

Before describing each of the functional components of the fluid handling system, several example configurations are now described. FIGS. 1a-c are block diagrams illustrating high-level configurations of an example fluid handling system. Each configuration is illustrated including a number of components or subsystems. Those subsystems are each described in more detail with reference to the following figures.

FIG. 1a is an illustration of an example configuration of a mobile system including a stationary filter, mobile tank, and end effector system. The stationary filter comprises optional pre-treatments, pre-treatment subsystem 10, CDI subsystem 12, mix and filter subsystem 14, output subsystem 16, stationary filter mechanical subsystem 18, and stationary filter electrical subsystem 20. The mobile tank comprises tank subsystem 22, tank liner subsystem 24, drug delivery subsystem 26, mobile tank mechanical subsystem 28, and mobile tank electrical subsystem 30. The end effector subsystem comprises the end effector subsystem 32.

In an example, the system may also manage waste fluids. The waste management system comprises a waste tank subsystem 34 and waste connection subsystem 36.

In an example, the system may use single-use bags for waste management. The waste bags hang on hooks 815 on the mobile tank and fill without any suction assistance. After the bags have reached capacity, the bags may be disconnected from the medical device and discarded.

In an example, the system may include waste bag mass sensors 818 and warnings for when bags are at or near capacity, waste bag hooks 815, and include a connection to suction on the waste bags and the vacuum pump to draw a vacuum in the bags 1100.

In other examples, the stationary filter may include an accumulator tank after the mix and filter Subsystem 14 and before the output subsystem 16. The tank can be configured to store at least about 10 liters of fluid that is available for rapid deployment. The tank may be constructed from a low-leaching polymer.

Advantages of this configuration include, but are not limited to capacitive deionization is a low power, low waste, light weight, and small form factor water purification method. Capacitive deionization retains endotoxins, adding another element of biological filtration. Capacitive deionization is regenerative, providing a long life and consistent efficiency. Waste and clean fluids are managed on one device. There is little risk in handling waste fluids as they are enclosed when being moved around the medical facility.

In an example, the system generates a batch of medical fluid using capacitive deionization and ultrafiltration. The capacitive deionization is accomplished by activated carbon electrodes. Activated carbon has bactericidal, antibacterial, antiviral, and virucidal properties. The carbon may be doped with additional substances to enhance properties, such as colloidal or organic silver. The system uses capacitive deionization for removing chemicals, ions, and endotoxins. There are redundant ultrafilters for biological purification and safety. The system may have sample ports for water quality testing. Using parallel CDI chambers, the system can run (e.g., continuously) without down-time. The deionization chamber may be replaced with reverse osmosis, electro dialysis, or charged ion beds. System can temperature control the fluid passing through the capacitive deionization chamber. System can use heated fluid from the capacitive deionization chamber for system disinfection, supplying warmed medical fluids to the point-of-use, and regeneration of electrodes. A medical irrigation instrument with attached filter in place of an aseptic connection. The system incorporates a drug delivery system. All purification and mixing of fluids is done in the stationary filter. The mobile tank system carries a prepared batch of medical fluid for medical application.

In an example, one mobile system manages both dean and waste fluids.

FIG. 1b is an illustration of an example configuration of a boom-mounted system. This example changes the system from a generator and reservoir (e.g., stationary filter, mobile tank, and end effector subsystem 32) to an on-demand system. In an example, the system may be mounted in a surgical boom arm.

The system comprises a stationary filter including optional pre-treatments 5, pre-treatment subsystem 10, CDI subsystem 12, mix and filter subsystem 14, output subsystem 16, modified stationary filter mechanical subsystem 18, and modified stationary filter electrical subsystem 20.

Tubing may be provided to transport the sterile fluid from the stationary filter to a surgical boom arm. Pipe material is a non-leaching polymer or stainless steel. The pipe material is necessarily flexible for the articulating boom arm.

In an example, boom arm output comprises drug delivery subsystem 26, modified mobile tank mechanical subsystem 28, and modified mobile tank electrical subsystem 30. The configuration is shown in FIG. 1b may also include an end effector subsystem 32 and may be connected to a medical device 7.

The example configuration may also include a waste tank subsystem 34, and a waste connection subsystem 36. This system may use single-use or reusable bags for waste management. The waste bags may hang on hooks 815 or sit in a space on the mobile tank and fill without any suction assistance. After the bags have reached capacity, the bags may be disconnected from the medical device and emptied. The bags may be used multiple times.

Waste bag mass sensors 818 may be used to issue warnings for when bags are at or near capacity. An example includes a connection to suction on the waste bags and the vacuum pump to draw a vacuum in the bags 1100. An example also includes a tube on the waste bag 817 and the waste suction connection 824 on the stationary filter to empty the contents of the waste bag.

Advantages of this configuration include, but are not limited to capacitive deionization is a low power, low waste, light weight, and small form factor water purification method. Capacitive deionization retains endotoxins, adding another element of biological filtration. Capacitive deionization is regenerative, providing a long life and consistent efficiency. Waste and clean fluids are managed on one device. There is minimal complexity for waste fluid management. The system provides ease of disposing the contents of the waste bags via the waste fluid connection on the stationary filter. There is a minimized risk in handling waste fluids, as they are enclosed when being moved around the medical facility

The system is a boom-mounted on-demand sterile fluid generator for medical applications. The system uses capacitive deionization for removing chemicals, ions, and endotoxins. Using parallel CDI chambers, the system can run (e.g., continuously) without down-time. Redundant ultrafilters for biological purification and safety. Example advantages of a boom mounted system include but are not limited to, no mobile tank to push around, less floor space used, and there is no need for a tank liner consumable.

The system generates on-demand medical fluid using capacitive deionization and ultrafiltration. The capacitive deionization is done by activated carbon electrodes. Activated carbon has bactericidal, antibacterial, antiviral, and virucidal properties. The carbon may be doped with additional substances to enhance properties, such as colloidal or organic silver. The system uses capacitive deionization for removing chemicals, ions, and endotoxins. There are redundant ultrafilters for biological purification and safety. The system has sample ports for water quality testing using parallel CDI chambers, the system can run (e.g., continuously) without down-time. The deionization chamber may be replaced with reverse osmosis, electro dialysis, or charged ion beds.

In an example, the system can control temperature of the fluid passing through the capacitive deionization chamber. The system can use heated fluid from the capacitive deionization chamber for system disinfection, supplying sterile, warmed medical fluids to the point-of-use, regeneration of electrodes, or supplying the medical fluid at a specified fluid temperature for the treatment. A medical irrigation instrument with attached filter in place of an aseptic connection. The system may also incorporate a drug delivery system. All purification and mixing of fluids is accomplished in the stationary filter. The boom arm connection provides an on-demand fluid option with tank storage.

FIG. 1c is an illustration of an example configuration of a transportable fluid management machine. This example comprises a transportable combined water purification and waste handling system that generates fluid on-demand The system comprises optional pre-treatments 5, pre-treatment subsystem 10, CDI subsystem 12, mix and filter subsystem 14, and output subsystem 16.

The system may also comprise a modified stationary filter electrical subsystem 20, tank subsystem 22, tank liner subsystem 24, drug delivery subsystem 26, mobile tank mechanical subsystem 18, mobile tank electrical subsystem 30, waste tank subsystem 34, and waste connection subsystem 36.

This system may employ a reusable tank for waste management. The tank may fill with waste fluid from the medical device. After the waste fluid tank is full, the mobile tank may be connected to the stationary filter. The waste pump may remove all of the fluid from the tank and then the disinfectant pump may push chemical disinfectant in to the tank. The mobile tank may be disconnected from the stationary filter and may be ready for another procedure.

In another example, the stationary filter comprises an accumulator tank after the mix and filter subsystem 14 and before the output subsystem 16. This tank may store at least 10 liters of fluid that may be available for rapid deployment. The tank may be constructed from a low-leaching polymer.

In another example, volume sensors 1115 may be provided on the waste tank with the ability to alert the user that the tank is at or near capacity. Vacuum pump 1100 may be provided on the waste tank with adjustable vacuum. Disinfection of the waste tank may be by using chemical disinfection 328. Disinfection of the waste tank may also be by using hot water from the CDI Chamber 110.

Advantages of this configuration include, but are not limited to capacitive deionization is a low power, low waste, light weight, and small form factor water purification method. Capacitive deionization retains endotoxins, adding another element of biological filtration. Capacitive deionization is regenerative, providing a long life and consistent efficiency.

Waste and clean fluids are managed on one device. No waste bags are needed. There is minimized risk in handling waste fluids as they are enclosed when being moved around the medical facility.

Before continuing, it should be noted that the example configurations described above are provided for purposes of illustration, and are not intended to be limiting. Other devices and/or device configurations may be utilized to carry out the operations described herein.

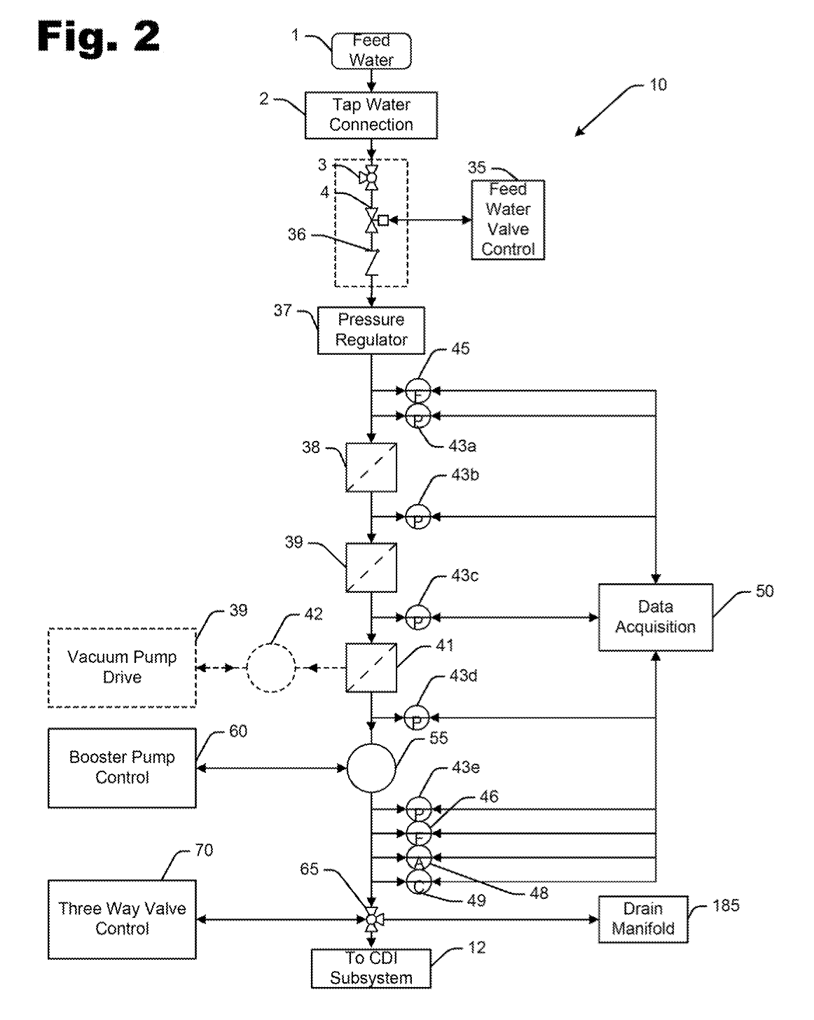

FIG. 2 illustrates an example pre-treatment subsystem 10 of the example combined water purification and waste handling system. This subsystem 10 receives feed water and pre-filters the feed water in preparation for subsequent filtration steps.

In an example, the subsystem 10 may include (optional) pre-treatment filters, (optional) booster pump, feed water 1, tap water connection 2, manual three-way valve 3, feed water valve 4, feed water valve control (e.g., circuits and logic) 35, backflow prevention valve 36, pressure regulator 37 (e.g., to set pressure greater than about 10 psi and less than about 150 psi), sediment filter 38 (e.g., having a pore size less than or equal to about 10 microns and replaceable at regular intervals or when needed), ultrafilter 39 (e.g., having a pore size less than or equal to about 0.22 microns, and replaceable at regular intervals or when needed), air removal filter 41, replaceable vacuum pump 42 (e.g., providing a vacuum level greater than or equal to about 5.0 inches of mercury), and vacuum pump control (e.g., circuits and logic) 39.

The subsystem 10 may also include pressure sensors 43a-e (e.g., pressure range of about 0-150 pounds per square inch, and resolution at least ±about 1 pounds per square inch), flow sensors 45, 46 (e.g., flow range of about 0-5 liters per minute, and resolution at least about ±0.1 liters per minute), air-in-line sensor 48 (e.g., having a range of about 1-1000 microliter bubbles, and a resolution of less than or equal to about 5 microliters), conductivity sensor 49 (e.g., having a conductivity range of about 0-2000 micro Siemens per centimeter, and resolution less than or equal to about ±5 micro Siemen per centimeter), data acquisition (e.g., circuits and logic) 50, booster pump 55 (e.g., capable of a pressure head greater than about 50 pounds per square inch, and flow rate greater than about 5 liters per minute), booster pump control (e.g., circuits and logic) 60, a valve (e.g., three-way valve) 65, valve control (e.g., circuits and logic) 70, and drain manifold 185.

The tap water connection 2 connects the device to a tap water source which seals the fluid path and introduces feed water 3 to the device. If the feed water 3 does not meet the EPA Primary Drinking Water standards, then pre-treatment filters 5 may be implemented to reduce contaminants in the water.

A manual valve 3 is placed after the tap water connection to enable the user to shut-off or divert water flow to the device during installation, maintenance, or safety reasons.

Feed water then flows through the automated feed water valve 4 which enables the machine to control the ingress of feed water. This may be closed during installation, maintenance, or when flow is not needed. The valve is controlled and driven by the feed water control (e.g., circuits and logic) 35.

The feed water enters a backflow prevention valve 35 which keeps fluid in the system from flowing back in to the feed water supply. The feed water then flows through a pressure regulator 37, which prevents over-pressure in the system. If the feed water is not sufficiently pressurized, a booster pump may be implemented in the feed water system before the tap water connection 2 to increase pressure. A flow sensor 45 is located after the pressure regulator to determine the flow of the feed water. The sensor output is captured by the data acquisition circuits and logic 50.

A pressure transducer 43a is in fluid communication with the fluid path after the regulator to monitor the inlet feed water pressure. The sensor output is captured by the data acquisition circuits and logic 50.

The feed water then enters a sediment filter 38 with pore size less than or equal to about 10 micron. This filter reduces chemical and biological contaminants and prevents large particles from fouling downstream filters. The sediment filter is replaceable at various intervals.

A pressure transducer 43b is located after the sediment filter. The pressure drop through the sediment filter, as referenced to the pressure transducer 43a, is monitored over time to determine the life of the filter. Fouling in the filter causes the pressure drop to increase. The sensor output is captured by the data acquisition circuits and logic 50.

The feed water then flows through an ultrafilter 39 with pore size less than or equal to about 0.2 micron. The filter removes bacteria, virus, and endotoxin from the feed water. The ultrafilter is replaceable at various intervals.

A pressure transducer 43c is in fluid communication with the feed water after the ultrafilter 30. The pressure drop across the ultrafilter, as referenced to the pressure transducer 43b, is monitored over time to determine the life of the filter. The sensor output is captured by the data acquisition circuits and logic 50. The feed water then flows through an air removal filter 41 to remove air and gas. The air removal filter is replaceable at various intervals.

A vacuum pump 42 may be attached to the air removal filter to enhance air removal by drawing a vacuum in the filter. The vacuum pump is driven and controlled by the vacuum pump drive circuits and logic 39.

A pressure transducer 43d is in fluid communication with the feed war after the air removal filter 41. The pressure drop across the air removal filter, as referenced to the pressure transducer 43c, is monitored over time to determine the life of the filter. The sensor output is captured by the data acquisition circuits and logic 50.

The feed water then enters a booster pump 55, which may be positive displacement or centrifugal pump, in order to increase the pressure of the fluid stream after the pressure drops in the degassing, sediment, and ultra-filters. The booster pump is driven and controlled by the booster pump control (e.g., circuits and logic) 60.

A pressure sensor 43e is located after the booster pump to determine the pressure of the water stream. The sensor output is captured by the data acquisition circuits and logic 50.

A flow sensor 46 is in-line with the flow to determine the flow rate of feed water. The sensor output is captured by the data acquisition circuits and logic 50.

An air-in-line sensor 48 measures the quantity of air in the water stream. The sensor output is captured by the data acquisition circuits and logic 50.

A conductivity sensor 49 is located before the valve 65 in order to measure the ion content in the water stream. The sensor output is captured by the data acquisition circuits and logic 50.

The treated water then enters an automated three-way valve 65. The valve is driven and controlled by the valve control (e.g., circuits and logic) 70.

Logic may be configured as follows. The flow 46 and pressure 43e sensors provides feedback for the booster pump. If flow or pressure is too low, the booster pump input may be increased. If the flow or pressure is too high, the booster pump input may be decreased. If the processor determines the water needs to be diverted to drain, the valve is activated and flow is diverted to the drain manifold. Otherwise, the water flows to the CDI Chamber Subsystem.

The system may be put in an installation or maintenance mode where water is diverted to the drain by the valve 65 for a predetermined period of time or until satisfying certain pressure, flow, or air parameters.

In other examples, an additional booster pump is provided before entering the tap water connection 2. In the event the feed water pressure is low, a booster pump may be provided to get adequate flow through the pre-treatment subsystem 10. With additional filtering pre-treatment before entering the tap water connection. If the water has a high presence of a particular chemical or particulate, additional pre-treatment before the system may be implemented Without the air removal filter 41. This filter may not be implemented and may be an optional pre-treatment. With a shutoff valve rather than a valve 65 and drain connection 185 at the end of the subsystem.

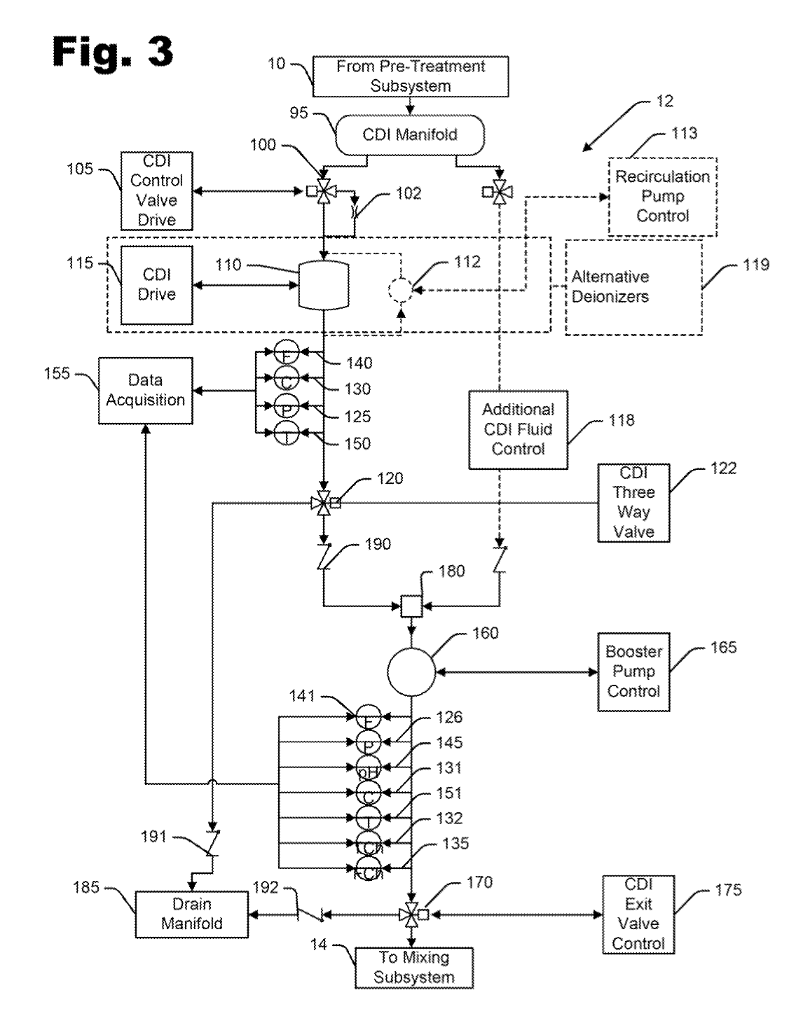

FIG. 3 illustrates an example capacitive deionization (CDI) subsystem 12 of the example combined water purification and waste handling system. The CDI subsystem 12 receives the output of the pre-treatment subsystem 10 and then removes ions and chemicals from the water.

In an example, the CDI subsystem 12 includes CDI manifold 95, CDI control valves 100, CDI flow restrictor 102 (e.g., flow rate reduced at least 50% when compared to high flow fluid path), CDI control valve drive 105, CDI chamber 110 (e.g., flow rate at least about 250 milliliters per minute while purifying to about 1.0 micro Siemens per centimeter), CDI drive 115, Additional CDI fluid circuits 118, alternative deionizers 119, CDI valve(s) 120, CDI valve drive 122, pressure sensors 125, 145 (e.g., pressure range of about 0-150 pounds per square inch, and resolution less than or equal to about ±1 pounds per square inch), conductivity sensors 130, 131 (e.g., conductivity range at least about 0-200 micro Siemens per centimeter, and resolution less than or equal to about ±0.25 micro Siemens per centimeter).

In an example, the CDI subsystem 12 may also include total chlorine sensor 132 (e.g., range of about 0-20 parts per million, and resolution less than or equal to about 0.1 parts per million), free chlorine sensor 135 (e.g., range of about 0-20 parts per million, and resolution less than or equal to about 0.1 parts per million), flow sensors 140, 141 (e.g., flow range of about 0-5 liters per minute, and resolution less than or equal to about ±0.1 liters per minute), pH sensor 145 (e.g., pH range of about 0-14 pH units, and resolution less than or equal to about ±0.25 pH units), temperature sensor(s) 150, 151 (e.g., temperature range of about 0-100° C., and resolution less than or equal to about ±1° C.), data acquisition (e.g., circuits and logic) 155, booster pump 160 (e.g., pressure head capable of greater than about 50 pounds per square inch, and flow rate greater than about 5 liters per minute), booster pump control (e.g., circuits and logic) 165, CDI exit valve 170, CDI exit valve control 175, deionized water manifold 180, drain manifold 185, and check valves 190, 191, 192.

The pre-treated water enters the CDI subsystem 12 from the pre-treatment subsystem 10. The pre-treated water enters the CDI manifold 95 that divides the flow in to one or more fluid paths. One fluid path is illustrated in the figure, but zero or more CDI fluid circuits 118 comprising the fluid circuit description below may be added.

In example, fluid enters the CDI control valve 100. The control valve is automated and has three states: no flow, low flow, or high flow. The CDI control valve drive circuits and control 105 operate the state of the valve and drive movement. In the no flow state, the valve blocks flow to the remainder of the CDI fluid circuit. This may occur when the machine is not in use or during maintenance. In the low flow state the valve diverts flow through the CDI flow restrictor 102 and in to the CDI chamber 110. This may occur during regeneration of the CDI chambers. In the high flow state, the valve diverts flow directly to the CDI chamber 110. This may occur in a deionizing mode, maintenance, filter replacement, or regeneration.

In the CDI chamber 110 the fluid is deionized. The deionization chambers are made from carbon material (activated carbon fiber, granular activated carbon, block granular activated carbon, aerogel, reticulated vitreous carbon, etc.). Water flows through the electrodes. Two electrodes of opposite charge generate an electromagnetic field where ions and charged chemicals are attracted to either the positive or negative electrode. The charge is generated by connection with a DC power supply. A differential voltage of between about 1.2 and 10 Volts is applied to the electrodes. The voltage may vary, depending on the position in the electrode stack. The electrodes may be encased in a non-leaching polymer housing. The electrodes are spaced by a polymer mesh or spacer. The spacing between electrodes may vary with position in the stack.

The CDI chamber voltage is driven and controlled by the CDI drive (e.g., circuits and logic) 115. After the fluid leaves the CDI chamber, four sensors monitor physical properties of the water: pressure 125, conductivity 130, temperature 150, and flow 140. The sensor output is captured by the data acquisition circuits and logic 155.

After the sensors, the CDI valve 120 diverts flow between two paths. The first path is through a check valve 191 and in to the drain manifold 185. The check valve prevents fluid intended for disposal from re-entering the CD fluid circuit. The second path is through a check valve 190 and in to the deionized water manifold 180, where water continues through the fluid circuit of the stationary filter system to the mixing subsystem 14. The check valve prevents water from another CDI fluid circuit from entering the fluid circuit.

After the deionized water manifold 180, the water passes through a booster pump 160. The booster pump control (e.g., circuits and logic) 165 drive the booster pump 160. The booster pump 160 increases the pressure of the fluid in the system after the CDI chambers.

In an example, the deionized water then enters sensors. Flow sensor 141 determines the flow of the water. Pressure sensor 126 determines the pressure of the water. A pH Sensor 145 determines the acidity/alkalinity of the water. Conductivity sensor 131 determines the ion/chemical content and conductivity of the water. Temperature sensor 151 determines the temperature of the water. Total chlorine sensor 132 determines the total amount of chlorine in the water. Free chlorine sensor 135 determines the amount of free chlorine in the water.

The data acquisition (e.g., circuits and logic) 155 converts data from analog to digital and communicates the measurements to the processor.

The CDI exit valve 170 diverts flow between two paths. The first path is through a check valve 192 to the drain manifold 185. The check valve prevents fluid intended for disposal from re-entering the CDI fluid circuit. The second path is to the Mixing Subsystem. The CDI exit valve 170 is controlled by the CDI exit valve control (e.g., circuits and logic) 175.

In an example, the CDI control valve 100 diverts water through either the high or low flow fluid paths, depending on the needs of the system. The CU drive (e.g., circuits and logic) 115 provides power to the electrodes. This may be in sequential steps or all at about the same time.

Based on the measurements from the deionized water output sensors 125, 130, 140, 150, the power to the CDI chamber drive may be manipulated by the CDI drive (e.g., circuits and logic) 115. Until the deionized water satisfies the specification, as determined by the data from the sensors 125, 130, 140, 150, the CDI valve 120 diverts flow to the drain manifold 185.

After the deionized water satisfies specification, as determined by the data from the sensors 125, 130, 140, 150, the CDI valve 120 diverts flow to the deionized water manifold 180. If at any point the water does not satisfy specification, then the CDI valve 120 is switched back to the drain manifold exit position.

After the electrodes reach capacity (electrode surface is covered so that no more ions/chemicals may attach) and the specification is no longer being satisfied, the CDI valve 120 immediately diverts the flow to the drain manifold 185 and the CDI chamber 110 goes in to regeneration mode. During regeneration mode, the CDI drive (e.g., circuits and logic) 115 manipulate power to the electrodes causing ions/chemicals to desorb from the electrode surfaces. The waste flow is diverted to the drain manifold 185 by the CDI valve 120. During regeneration, the flow path may be diverted by the CDI control valve 100 to the high flow or low flow 102 circuits, depending on the regeneration needs.

After regeneration criteria have been satisfied (may be electrode current, electrode voltage, conductivity of effluent, etc.), the system may either go in to a rest mode (no flow) or return to a purification mode.

In another example, there may be one or multiple CDI fluid circuits, drive circuits, and logic 118. An accumulator tank may be added in the CDI circuit. A tank of less than about 100 liter volume may be filled with sterilized feed water (from the Pre-Treatment subsystem). The CDI circuit may draw from the tank, deionize the water, and circulate back in to the tank until the conductivity, chlorine, chloramine, and pH of the bulk fluid was at an acceptable level. The fluid may then be pumped in to the Deionized Water Manifold.

In another example, the CDI chamber may be replaced by an alternative deionizing system 119, including: reverse osmosis electrodeionization, electro dialysis, charged ion beds The CDI chamber electrode configuration may be flow-by electrodes instead of flow-through electrodes. The electrodes in the CDI chamber 110 may have current passed across each one, rather than from one electrode to the next across the gap (as a capacitor). The internal resistance of the electrode may use the electrode to heat up. When fluid flowed through the electrode, temperature may raise due to convective heat transfer from the electrode. Heating fluid above a certain temperature threshold may enable for disinfection of the system and more effective regeneration. Electrodes may be used to heat fluid during purification to generate a sterile fluid at a specified temperature.

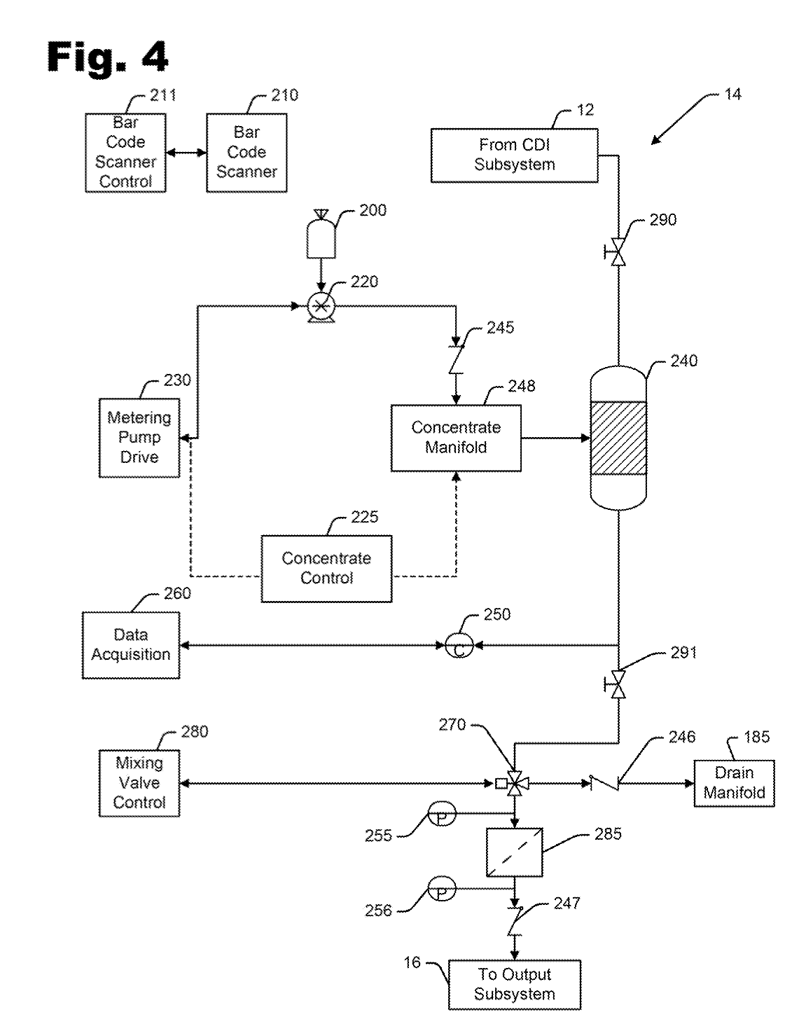

FIG. 4 illustrates an example mix and filter subsystem 14 of the example combined water purification and waste handling system. This subsystem combines concentrate with deionized water to form a fluid of specified concentration, and then removes bacteria, virus, and endotoxin from the fluid by ultrafiltration.

In an example, the mix and filter subsystem 14 may receive water from the CD subsystem 12 and may include one or more containers of concentration fluid 200 (e.g., volume greater than about 500 milliliters), barcode scanner 210, bar code scanner control (e.g., circuits and logic) 211, metering pump 220 (e.g., flow rate range of about 0-100 milliliters per minute, and pressure head capable of greater than or equal to about 50 pounds per square inch), additional concentration circuits 225, metering pump control (e.g., circuits and logic) 230, mixing chamber 240, check valves 245, 246, 247, concentration manifold 248, conductivity sensor 250 (e.g., conductivity range of about 0-40,000 micro Siemens per centimeter, and resolution less than or equal to 100 micro Siemens per centimeter), data acquisition 260, mixing valve 270, mixing valve control 280, drain manifold 185, sample port 290, 291, and ultrafilter 285 (e.g., having a pore size less than or equal to about 0.22 microns).

Water (dilutant) from the CDI subsystem 12 enters the mix and filter subsystem 14. Fluid from the CM subsystem 12 passes through a sample port 290. The sample port 290 may be a push button port with a spout the can be easily accessed for filling a sample container. This may be implemented for capturing samples of fluid for water quality tests. The dilutant enters the mixing chamber 240. In an example, mixing chamber 240 may be a separate volume or a stretch of tubing.

A barcode scanner 210 convenient to the user is employed to read critical information from the concentration fluid labeling. Example information may include, but is not limited to, fluid description, concentration, lot number, or expiration date. The bar code scanner is controlled by the bar code scanner control (e.g., circuits and logic) 211.

Concentrate is added to the mixing chamber by one or more of the concentrate circuits 225. Each concentrate circuit 225 includes the following. One or more containers of medical fluid concentrate 200 are connected to the metering pump 220. The concentrate may be of injection, irrigation, hemodialysis, hemofiltration, hemodiafiltration, or other medical fluids. The metering pump 220 apportions the concentrate fluid 200 in to the mixing chamber 240. The metering pump 220 is driven and controlled by the metering pump control (e.g., circuits and logic) 230. The concentrate exits the metering pump 220 and enters a check valve 245. The check valve 245 prevents concentrate or dilutant from flowing back in to the metering pump 220 and concentrate container 200.

After exiting the check valve 245, the concentrate enters the concentrate manifold 248. The concentrate manifold 248 accepts one or more concentrate fluid circuits 225 and generates a fluid path in to the mixing chamber 240. The concentrate and dilutant enter the mixing chamber 240 where the fluids are mixed.

After exiting the mixing chamber 240, the fluid passes a conductivity sensor 250 for a check of the fluid's conductivity against known values for the desired fluid and concentrate. Data acquisition circuits and logic 260 capture the conductivity measurement from the sensor and store the value in memory for access by the system logic.

The fluid passes through a sample port 291. The sample port 291 may be a push button port with a spout the can be easily accessed for filling a sample container. This may be implemented for capturing samples of fluid for water quality tests.

The fluid exits the sample port 291 and enters a valve, the mixing valve 270. In an example, the valve 270 has two positions. The first directs the flow to the drain manifold 185. The second directs the flow to the output subsystem 16.

After exiting the mixing valve 270, the fluid passes through a check valve 246 before entering the drain manifold 185. This valve prevents fluid intended for disposal from re-entering the subsystem 14. After exiting the mixing valve 270, the fluid enters the ultrafilter 285, the fluid enters the ultrafilter, which removes bacteria, virus, and endotoxin. The diameter of the pores is less than about 0.22 micron. The fluid exits the ultrafilter 285 and enters a check valve 247, which prevents fluid from being pushed back in to the Mix and Filter Subsystem.

Example logic may be implemented at least in part as computer software and/or firmware and/or hardwired. The system logic communicates with the flow sensors in the CD subsystem 140 or 141 to determine the correct flow rate of concentrate 200, and by calculation the speed of the metering pump 220. The flow rate of the concentrate fluid is based on the measured flow rate of the dilutant, the desired concentration of fluid, and the concentration of the concentrate (from the barcode scanner logic).

The system logic reads the critical information from the barcode scanner 210 and logic 211. This data is logged and used to determine the appropriate concentration of fluid, and therefore the speed of the metering pump.

The conductivity of the fluid, as measured by the conductivity sensor 250, is compared against known values for the desired concentration of fluid. If the conductivity is in the acceptable range, the mixing valve 270 enables the fluid to continue to the output subsystem 16. If the fluid fails the conductivity test, the mixing valve 270 directs the fluid to the drain manifold 185. If the fluid is found unacceptable for a significant period of time, the user is alerted. The default position of the mixing valve 270 is open to the drain manifold 185.

Other example configurations may include any sort of optical or RFD recognition of the concentrate fluid instead of a barcode scanner 210, 211. This may include QR codes, 1D barcodes, visual symbols by image detection, etc. This subsystem may be skipped if the purpose of the machine is solely to generate sterile water. The check valve 245, concentrate manifold 248, and mixing chamber 240 may be one integrated physical unit. The addition of a booster pump may be provided before the ultrafilter in order to boost pressure and flow through the filter and then the Output Subsystem.

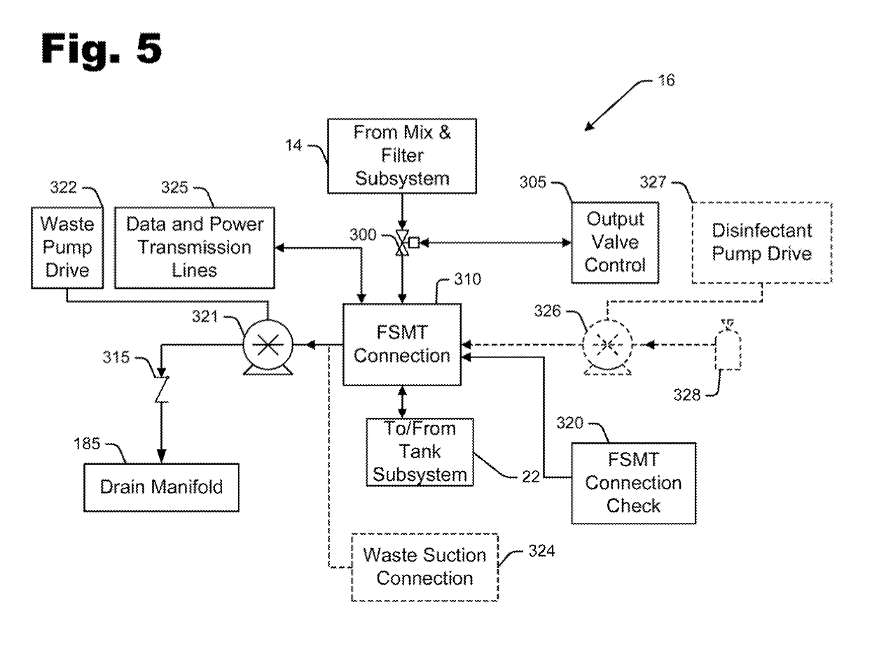

FIG. 5 illustrates an example output subsystem 16 of the example combined water purification and waste handling system. The output subsystem connects to the mobile tank for transferring the fluid from the stationary filter to the mobile tank.

In an example, the output subsystem may receive sterile fluid from mix and filter subsystem 14. The output subsystem 16 may include output valve 300, output valve control (e.g., circuits and logic) 305, filter station to mobile tank connection (FSMT connection) 310, check valve 315, FSMT connection check (e.g., circuit and logic) 320, data and power transmission lines 325, waste pump drive (e.g., circuits and software) 322, waste pump 321, waste suction connection 324, disinfectant pump 326, disinfectant pump drive circuits and software 327, disinfectant 328, and drain manifold 185.

In an example, the waste pump 321 may be configured to operate in a flow range of about 0 to 2,000 mL per minute. The maximum pressure head may be greater than or equal to about 5 pounds per square inch. The disinfectant pump 326 may be configured to operate in a flow range of about 0 to 1,000 mL per minute. The maximum pressure head may be greater than or equal to about 5 pounds per square inch.

Sterile fluid enters the output subsystem 16 from the mix and filter subsystem 14. The fluid enters the output valve 300. The valve controls the flow of fluid between the stationary filter and the mobile tank. The output valve control circuits and logic 305 control and drive the valve based on commands from the control logic.

The fluid then enters the FSMT connection 310. The FSMT connection 310 includes the following. Sterile fluid connection directs sterile fluid from the filter station in to the mobile tank. A used fluid connection directs fluid remaining in the mobile tank back to the stationary filter. An electrical power connection for charging a battery on the mobile tank. A data connection communicates data and status from the mobile tank to the stationary filter and vice versa. A means for determining the FSMT connection is complete, FSMT connection check circuit and logic. A means for protecting the connections exposure to contaminants when not in use.

Fluid from the used fluid connection flows through a check valve 315 and in to the drain manifold 185. The FSMT connection check circuit and logic 320 detects the presence and correct relative placement of the mobile tank in relation to the stationary filter. The data and power lines 325 establish digital communication and power transmission means between the stationary filter and mobile tank. The battery on the mobile tank is charged with power from these lines.

In an example, a disinfectant fluid connection may be provided to conduct disinfectant fluid from the stationary filter to the mobile tank. Waste fluid from the waste fluid connection of the FSMT connection 310 is pumped out of the tank by the waste pump 321 and flows through a check valve 315 and in to the drain manifold 185. The waste pump is driven and controlled by the waste pump drive circuits and software 322. The FSMT connection check circuit and software 320 uses a means to detect the presence and correct relative placement of the mobile tank in relation to the stationary filter.

The data and power lines 325 establish digital communication and power transmission means between the stationary filter and mobile tank. The battery on the mobile tank is charged with power from these lines.

Logic may be implemented at least in part as computer software and/or firmware and/or hardwired. The logic waits for confirmation from the FSMT connection check circuit and logic to open the valve for fluids, as well as make the electrical power and data connections.

In an example, the control enables the user to turn on the waste pump 321 to draw waste fluid through the waste suction connection 324. The software is able to determine that the fluid sources have been emptied and turns off the waste pump 321. The waste pump 321 may also be turned off by the user. The FSMT connection check circuit and software 320 and system software determine when the disinfectant pump drive circuits and software 327 may start and stop the disinfectant pump 326 and waste pump 321.

In other examples, variations may include a UV light may be added in the protective covering over the FSMT connection 310. The UV light neutralizes bacteria and virus, and denatures endotoxin prior to connection with the mobile tank.

In a further example, waste suction connection 324 may connect to a waste fluid bag on the mobile tank. Waste fluid may be drawn from the waste suction connection 324 through the waste pump 324. The waste suction connection 324 may be a form of fluid connection that may be off-the-shelf or proprietary.

In an example, a disinfectant system is used to chemically disinfect the mobile tank waste tank. The chemical disinfectant 328 is stored in an off-the-shelf or proprietary container. The disinfectant 328 is connected by means of a tube or other fluid conducting element to the disinfectant pump 326. The disinfectant pump 326 drives the disinfectant in to the FSMT connection 310.

In another example, the CDI filter may be used as a heating element to heat incoming fluid and the high temperature (greater than about 6° C.) fluid may be used to disinfect the mobile tank waste tank, rather than a chemical disinfectant.

FIG. 6 illustrates an example filter station mechanical subsystem 18 of the example combined water purification and waste handling system. Subsystem 18 provides a means for supporting all other subsystems and attaching to the building where the filter station installed.

The filter station mechanical subsystem includes structure 330, access panel 340, drain manifold 185 (e.g., flow rate to drain at least about two liters per minute), check valve 350, and drain connection 360.

The structure 330 enables points of connection to support the other subsystems in the stationary filter. The access panel 340 enables access to the subsystems for installation and maintenance, and may also protect the subsystems from the environment around the system. The drain manifold 185 provides a point of connection for all the subsystems that direct fluid to, the drain. The drain manifold 185 may have many inputs and one output to the check valve 350. The check valve prevents drain fluids from re-entering the system. The drain fluid exits the check valve 350 and enters the drain connection 360 where the fluid is flushed in the facility's drain.

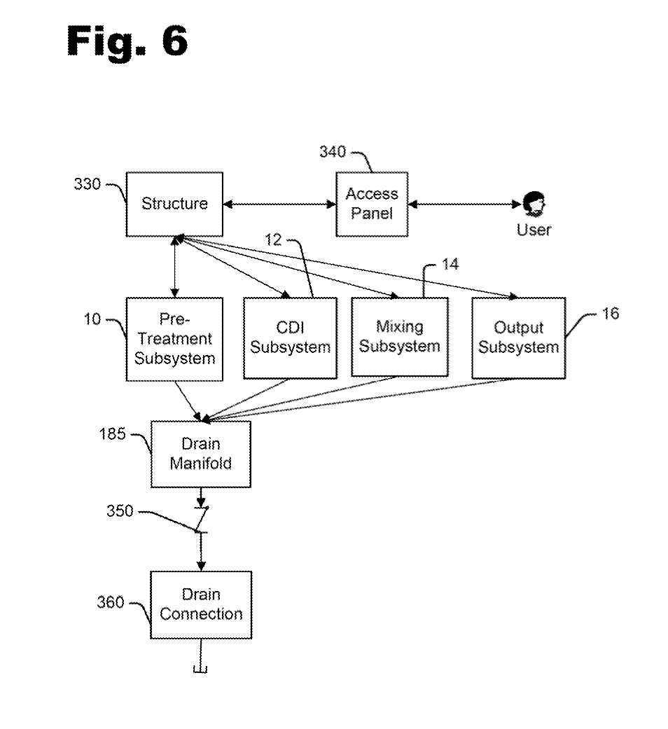

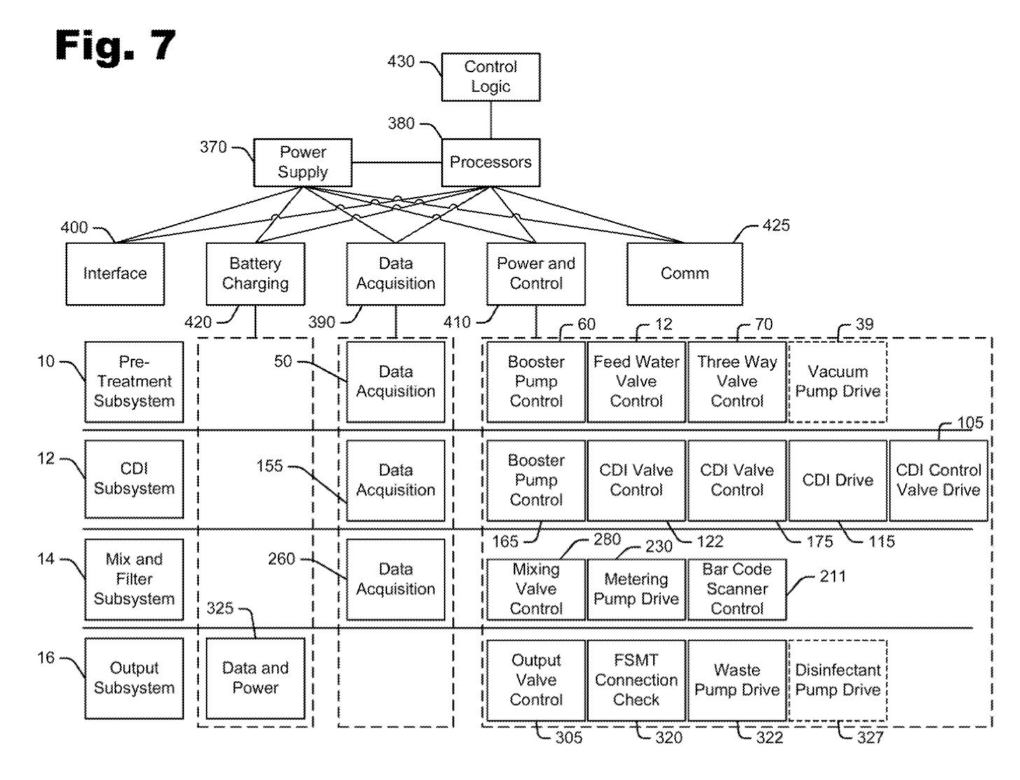

FIG. 7 illustrates an example filter station electrical subsystem 20 of the example combined water purification and waste handling system. Subsystem 20 executes logic, acquires data from mechatronic sensors, charges the mobile tank battery, powers actuators, and provides a user interface.

In an example, the filter station electrical subsystem includes AC/DC a medical grade power supply and power connection 370, processors 380, data acquisition circuits 390, interface (e.g., a touchscreen display and Graphical User Interface or GUl) 400, power and control 410, battery charging circuit 420, communication transceiver 425, and control 430.

In an example, power enters the power supply 370 and is converted from alternating current to direct current in a manner compliant with medical device standards. The processors 380 are powered by the DC power from the power supply 370. The processors 380 execute the logic and safety measures to run the device based on the control 430.

The data acquisition circuits 390 are powered by the power supply 370. The circuits 390 convert analog information from the sensors in the system to digital signals for the processors 380.

The interface 400 is powered by the power supply 370. In an example, the display of interface 400 may output graphics as instructed by the processor 380 and control 430 to generate the graphical user interface. The touchscreen 400 communicates physical touch commands from the user to the processor 380. The control 430 instructs the system based on the user commands.

The power and control 410 are powered by the power supply 370. These circuits energize the actuators, receive feedback from the actuators, and control the position of the actuators. The battery charging circuit 420 is powered by the power supply 370. This circuit charges the battery in the Mobile Tank. In an example, the control 430 is a set of instructions that run on the processor 380.

A communications component (e.g., a wireless communication transceiver) 425 may send and/or receive data between the Mobile Tank, Stationary Filter, and local area network. The communications component may be powered by the power supply 370 and communicates information to and from the processor 380.

The pressure drop across (difference between pressure before and pressure after) a filter is known at a given flow rate. The system measures the pressure drop across all filters (sediment filter, ultrafilter, air removal filter) and compares to known values. If the system observes that the pressure drop approaches the maximum acceptable value, the user is notified that filter replacement may be needed shortly. If the pressure drop exceeds the acceptable value, the Stationary Filter may stop functioning until the filters are replaced.

The device may be connected to the Internet and send a signal to the manufacturer when the pressure drops approach the acceptable limit. This may trigger an automated reorder of the filter and shipment to the user.

The system maintains usage data. This data may be stored on the Stationary Filter, Mobile Tank, on an email account, on a local device via Ethernet, a server location, remote data storage via cell phone connection, or an EMR database.

The data may include the following. Batch details, including time- and batch-stamped measurements for all sensors. Case details, including user profile selection, patient name, end effectors used, flow rates and pressures from the mobile tank, type of fluid used, and amount of fluid used. System errors, faults, messages. Maintenance status, including filter life, total volume of fluid purified, number of batches generated, etc.

The system may reorder concentrate as concentrate is used. The user may select the volume to fill in the tank, up to the tank limit volume. This enables the user to select an amount of fluid that is needed, rather than the maximum volume. The user may select the temperature of the water, up to about 40° C. This enables the use to select a fluid temperature best suited for the patient and the therapy provided.

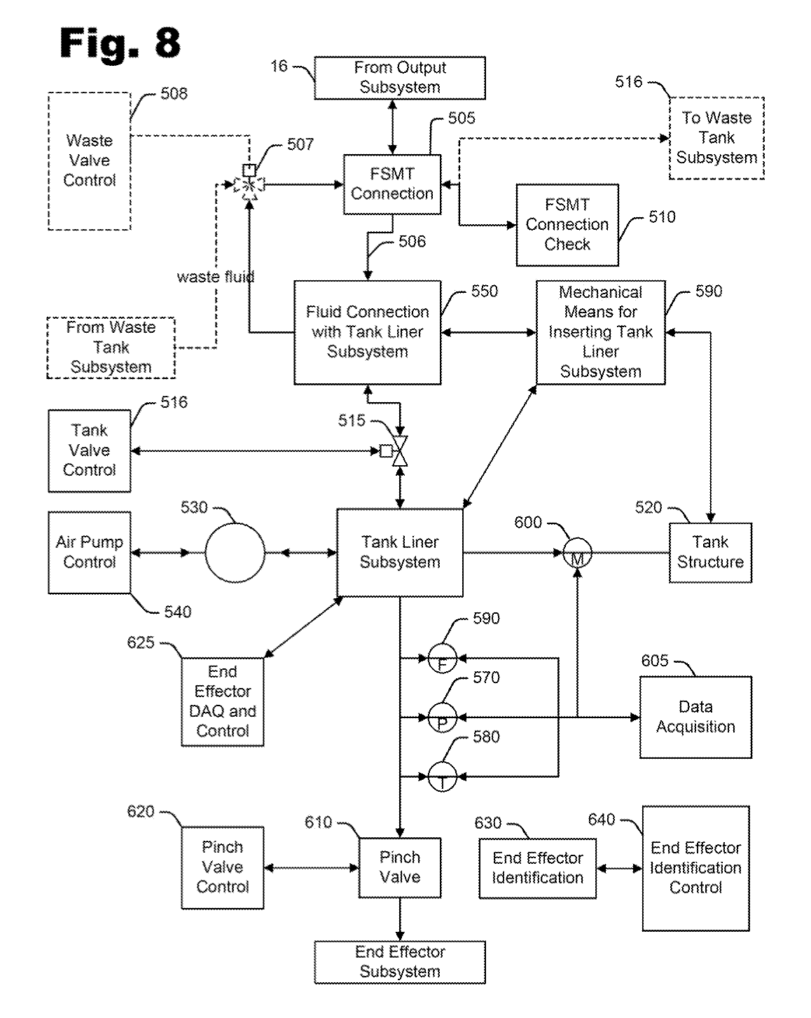

FIG. 8 illustrates an example tank subsystem 22 of the example combined water purification and waste handling system. Subsystem 22 enables connecting the mobile tank to the stationary filter, inserting a tank liner, and pressurizing the fluid in the tank.

The tank subsystem 22 receives fluid from output subsystem 16. An example tank subsystem 22 may include a filter station to mobile tank connection (FSMT connection) 505, dean fluid 506 line, waste valve 507, waste valve control (e.g., circuits and logic) 508, FSMT connection check (e.g., circuit and logic) 510, tank valve 515, tank valve control (e.g., circuits and logic) 516, tank structure 520, air pump 530 (e.g., pressure head at least about 20 pounds per square inch, and flow rate at least about 5 liters per minute), air pump control (e.g., circuits and logic) 540, fluid connection with tank liner subsystem 550, mechanical means for inserting tank liner subsystem 24, pressure sensor 570 (e.g., pressure range of about 0-100 psi, resolution less than or equal to about ±1 pound per square inch), temperature sensor 580 (e.g., temperature range of about 0-100° C., and resolution less than or equal to about ±1° C.), flow sensor 590 (e.g., flow range of about 0-5 liters per minute, and resolution less than or equal to about ±0.1 liters per minute), mass sensor 600 (e.g., mass range of about 0-100 kilogram, and mass resolution less than or equal to about 0.1 kilogram), data acquisition circuits and logic 605, pinch valve 610, pinch valve control (e.g., circuits and logic) 620, end effector DAQ and control 625, end effector identification 630, and end effector control (e.g., circuits and logic) 640.

The FSMT connection 505 includes a sterile fluid connection, directing clean fluid 506 from the filter station in to the mobile tank. A used fluid connection, directing waste fluid 507 remaining in the mobile tank back to the stationary filter and subsequently the drain. An electrical power connection provides a means for charging a battery on the mobile tank. A data connection provides a means for communicating data and status from the mobile tank to the stationary filter and vice versa. A means for determining the FSMT connection is complete, FSMT connection check (e.g., circuit and logic) 510.

The tank provides a structure 520 in which the tank liner subsystem holds fluid. The structure 520 may be pressure rated to greater than 100 psi inside the tank.

The air pump 530 draws air from the surroundings and pumps air into the tank liner subsystem 24. The air pressurizes the tank liner to provide a motive force for moving the sterile fluid out of the tank liner subsystem 24.

The air pump control (e.g., circuits and logic 540 drive and control the air pump 530. The system logic reads the pressure sensor 570 and generates a closed-loop control system to maintain pressure in the tank liner subsystem 24.

There is a fluid connection with the tank liner subsystem 24. This fluid connection is in fluid communication with and between the FSMT connection 505 and the tank valve 515. The tank valve control (e.g., circuits and logic) 516 drive and control the tank valve.

After the fluid exits the tank valve 515, the fluid enters the tank liner subsystem 24. There is a mechanical means 560 for inserting and securing the tank liner subsystem 24. One or more pressure sensors 570 are in communication with the tank liner subsystem 24 to determine the pressure inside the tank liner. Data acquisition (e.g., circuits and logic) 605 capture the pressure measurement from the sensor and store the value in memory for access by the system logic.

One or more temperature sensors 580 are in communication with the tank liner subsystem 24 to determine the temperature of the sterile fluid. Data acquisition (e.g., circuits and logic) 605 capture the temperature measurement from the sensor and store the value in memory for access by the system logic.

One or more flow sensors 590 are in communication with the tank liner subsystem 24 to measure the flow from the tank liner subsystem 24 to the end effector subsystem 32. Data acquisition (e.g., circuits and logic) 605 capture the flow measurement from the sensor and store the value in memory for access by the system logic.

One or more mass sensors 600 are in communication with the tank liner to determine the mass of fluid in the, tank liner. Data acquisition circuits and logic 605 capture the mass measurement from the sensor and store the value in memory for access by the system logic.

A pinch valve 610 pinches off the flow between the end-effector subsystem 32 and the tank liner subsystem 24 to prevent flow from leaving the system before the user “turns on” the flow to the medical device. The pinch valve control 620 drive and control the pinch valve 610.

The end effector DAQ and control 625 provide a low voltage power source for end effectors and enable a data input as well. This enables controls, motor activation, etc. on the end effector device.

An end effector identification device 630 uses a marking or mechanical feature on the end effector subsystem 32 and identifies the device. This may be a barcode, RFID tag, or other optical or mechanical means for identifying the end effector. The system logic may use the identification to change operating parameters such as fluid pressure, flow rate, and temperature. The end effector identification control (e.g., circuits and logic) 640 control the identification device 630.

Logic may be implemented at least in part as computer software and/or firmware and/or hardwired. The logic waits for confirmation from the FSMT connection check circuit and logic to open the tank valve 575, which enables clean fluid 506 to flow in to and waste fluid 507 to exit the tank liner subsystem 24. The confirmed connection also enables the establishment of electrical power and data lines between the stationary filter and mobile tank.

There is a closed loop control system for maintaining the pressure in the tank liner subsystem 24. The air pump 530, pressure sensor 570, and air pump control (e.g., circuits and logic) 540 are used for the control system. The mass sensor 600 monitors the amount of fluid in the tank liner subsystem 24. If the fluid supply runs low during operation, the user is notified via the mobile tank user interface. The pinch valve 610 defaults to a closed position on the tank liner connection manifold tank liner subsystem 24. When the user instructs the device to flow and the end effector subsystem 32 is detected, the pinch valve is opened and sterile fluid flows from the tank liner to the end effector.

Other examples may include using a peristaltic pump instead of an air pump in order to generate a motive force for evacuating fluid from the mobile tank. A peristaltic pump tubing segment on either tank liner consumable or end effector consumable to push fluid to irrigation device, rather than a pressurized tank. A heater element may be provided for heating the fluid and maintaining a fluid temperature in the tank. A filtration circuit may be provided that filters (e.g., continuously) the fluid in the tank liner. An ultrafilter and pump are connected in series with input from and output to the tank. The pump runs continuously during operation to filter any bacteria, virus, or endotoxin and maintain the biologically quality of the sterile fluid. One or more UV light sources may be provided around the tank or in-line with the tubing. This provides a means for killing bacteria and virus, and denaturing endotoxin. A UV light may be added in the protective covering over the FSMT connection 505. The UV light neutralizes bacteria and virus, and denatures endotoxin prior to connection with the Stationary Filter.

In an example, waste fluid from the waste tank subsystem 34 is connected to the waste valve 507. The waste valve 507 is operated by thee waste valve control (e.g., circuits and logic) 508. When instructed by the software, the waste valve 507 adjusts to the position of allowing waste fluid from the waste tank subsystem 34 to enter the valve and exist to the FSMT connection 505.

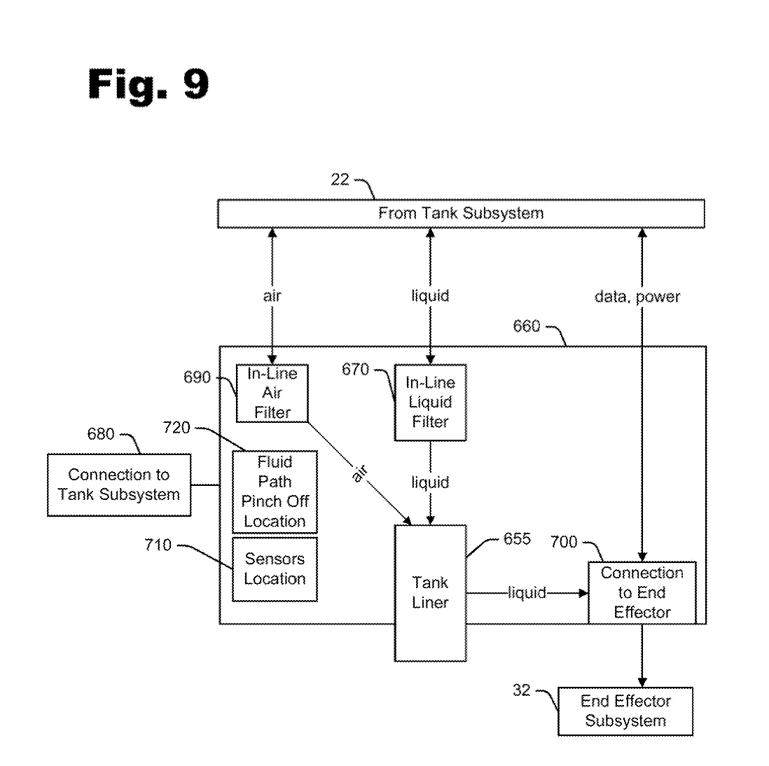

FIG. 9 illustrates an example tank liner subsystem 24 of the example combined water purification and waste handling system. Subsystem 24 is a multiple-use consumable tank liner which connects to the tank subsystem 22 and the end effector subsystem 32.

The tank liner subsystem may receive fluid from tank subsystem 22. An example tank liner subsystem 24 includes tank liner 655, connection manifold 660, in-line liquid filter 670 (e.g., having pore size less than or equal to about 0.22 micron), connection to tank subsystem 22, in-line air filter 690 (e.g., having pore size less than or equal to about 0.22 micron), connection to end effector 700, sensors location 710, fluid path pinch-off location 720

Fluid and air enter and exit the tank liner subsystem 24 from the tank subsystem 22. In an example, the tank liner 655 is a polymer bag suitable for containing about 10-100 liters of fluid. The tank liner 655 is attached to and in fluid communication with the connection manifold 660.

In an example, the connection manifold 660 provides the following connections. A fluid connection to the tank subsystem 22 for receiving sterile fluid in to the tank liner 655 and pumping out waste fluid from the tank liner 655. The in-line liquid filter 670 is an element of this connection. A drain fluid connection to the tank subsystem 22 for removing unused fluid in the tank liner. An air connection to the tank subsystem 22 for receiving pressurized air. The in-line air filter 690 is an element of this connection. An interface for tank subsystem sensors 710. This interface enables the pressure, flow, and temperature measurements to be made. A fluid connection 700 to the end effector subsystem 32 for transferring fluid to the end effector subsystem 32. A data and power connection 700 to the end effector subsystem 32. This enables the mobile tank to power and read data from the end effector. A fluid path pinch-off location 720 provides a place for the pinch valve in the Tank Subsystem to pinch off flow to the end-effector subsystem 32.

The in-line liquid filter 670 may remove bacteria, virus, and endotoxin from the sterile fluid moving in to the tank liner. This filter may be implemented as a safety precaution due to a non-aseptic connection between the stationary filter and mobile tank. The pore size is less than or equal to about 5 microns. The in-line air filter 690 removes bacteria, virus, and endotoxin from the air used to pressurize the tank liner. The pore size is less than or equal to about 5 microns.

In other examples, a filtration circuit may be provided that filters (e.g., continuously) the fluid in the tank liner. An ultrafilter and pump are connected in series with input from and output to the tank. The pump runs (e.g., continuously) during operation to filter any bacteria, virus, or endotoxin and maintain the biologically quality of the sterile fluid. A tank filter may be provided that transmits UV light. The UV light enters the fluid and kills bacteria and virus, and denature endotoxins.

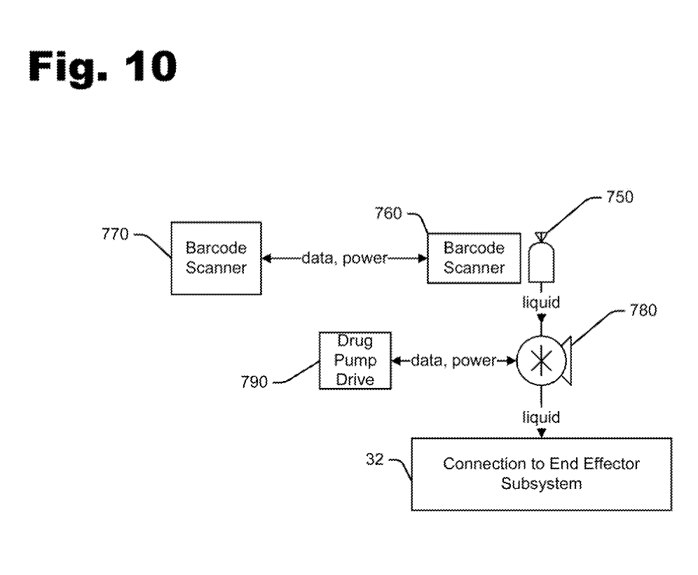

FIG. 10 illustrates an example drug delivery subsystem 26 of the example combined water purification and waste handling system. This subsystem provides a means for injecting a drug in to the sterile fluid.

An example drug delivery subsystem 26 may include drug container 750, barcode scanner 760, barcode scanner (e.g., circuits and logic) 770, drug pump 780 (e.g., flow rate range of about 0-100 milliliters per minute, and pressure head greater than or equal to about 50 pounds per square inch), drug pump control (e.g., circuits and logic) 790, and connection to end effector subsystem 32.

In an example, the drug container 750 is filled with any pharmaceutical or sterile additive to be diluted in the sterile fluid. The drug container is connected to the drug pump 780. The drug pump precisely meters the drug in to the end effector subsystem 32. The drug pump drive 790 (e.g., circuits and logic) control the rate of the drug pump 780. A barcode scanner 760 convenient to the user is employed to read critical information from the drug labeling. Example information may include, but is not limited to drug description, lot number, or expiration date. The barcode scanner (e.g., circuits and logic) 770 control the barcode scanner. A connection to the end effector subsystem 32 enables the drug to be pumped in to the sterile fluid and in to the medical device.

Logic may be at least in part as computer software and/or firmware and/or hardwired. The system logic communicates with the flow sensors in the tank subsystem 22 to determine the correct speed of the drug pump. The flow rate of drug out of the syringe pump is based on the measured flow rate of the sterile fluid, the desired concentration of drug in the sterile fluid, and the concentration of the drug. The system logic reads the critical information from the barcode scanner and logic. This data is logged and used to determine the appropriate concentration of sterile fluid, and therefore the speed of the syringe pump. The system runs a priming mode to get the drug to the connection to the end effector subsystem 32. This may be an automated mode or semi-automated with the assistance of the user.

Other examples may include a syringe pump or metering pump for the drug pump. Any sort of optical or REID recognition of the concentrate fluid may be implemented, e.g., instead of a barcode scanner.

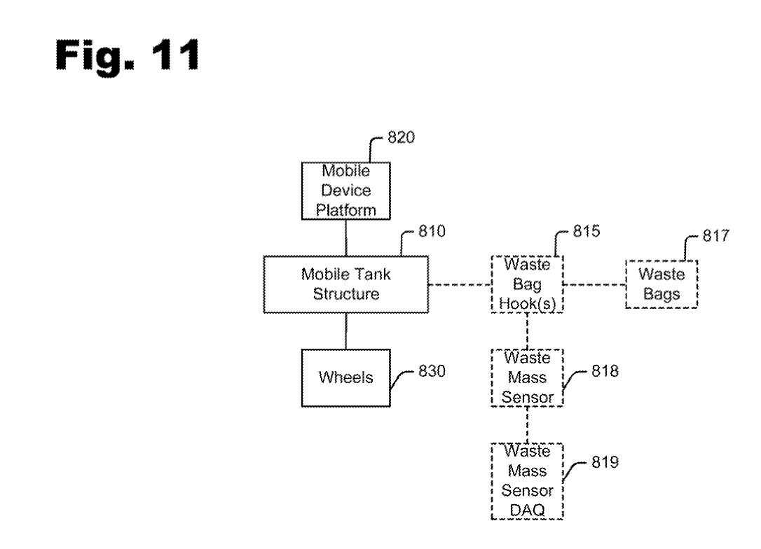

FIG. 11 illustrates an example mobile tank mechanical subsystem 28 of the example combined water purification and waste handling system. Subsystem 28 transports the mobile tank and provides a structure for attachment of all mobile tank subsystems.

An example mobile tank mechanical subsystem includes mobile tank structure 810, medical device platform 820, and wheels 830. The subsystem may also include one or more waste bag hooks 815, waste bag(s) 817, waste bag mass sensor 818, and waste bag mass sensor (e.g., circuits and software) 819.

The structure 810 provides a means for adequately supporting the mass of sterile fluid, all subsystems, and equipment stacked on the top of the device. The structure 810 provides a flat surface on the top of the mobile tank where a medical device may be placed. This device may be an arthroscopy pump, other surgical consoles, a hemofiltration machine, or a hemodialysis machine. The structure is supported by wheels 830 that enable the mobile tank to easily be pushed from one location to another across cords, door jams, elevator entrances, etc. The wheels have a break in order to stop the tank from rolling. Tank height, diameter, shape and location are determined in order to optimize risk of tipping over.

In an example, the structure 810 has waste bag hooks 815 to hang the waste bags 817. The waste bags 817 have a volume of one liter or more and have a hole in the structure to support a full bag hanging from a hook.

In another example, for large bags, the structure 810 may provide a space within which a waste bag 817 can rest and be supported while being filled.

For large waste bags 817, greater than three liters, the bags may have an attached tube with a connection for emptying the contents of the bag. The connection may be compatible with the waste suction connection 324 in the output subsystem 16. All entrances and exits from the waste bags 817 may have a means for stopping flow from the bag when the entrances/exits are disconnected.

The waste mass sensor 818 measures the mass in the waste bag. This may be a force sensor, strain gauge, optical sensor, or pressure transducer. The waste mass sensor DAQ (e.g., circuits and software) 819 convert the signal from the waste mass sensor 818 to digital data for the processor. The processor may determine when the waste bag is close to or at capacity and warn the user of such.

Other examples may include an automated drive system (comprising motor, gears, drive shaft, wheels, activation switch, etc.) that powers movement of the mobile tank.

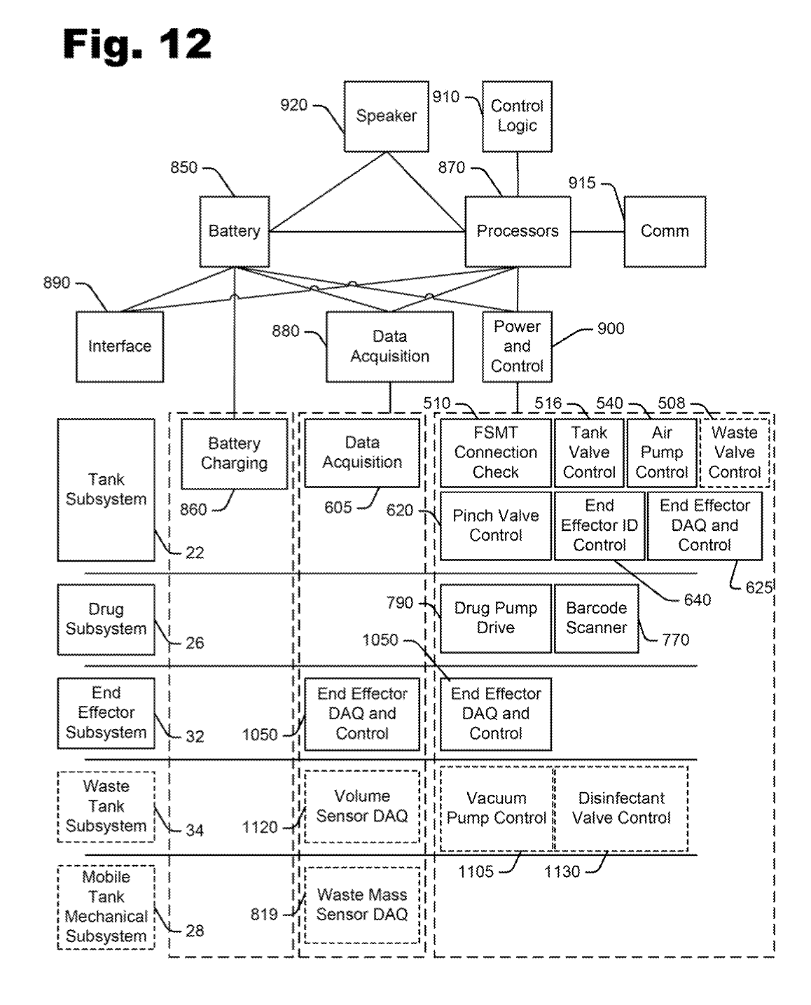

FIG. 12 illustrates an example mobile tank electrical subsystem 30 of the example combined water purification and waste handling system. Subsystem 30 provides the power and communication infrastructure for the mobile tank.

An example mobile tank electrical subsystem 30 includes battery 850, battery charging circuit 860, processor 870, data acquisition (e.g., circuits and logic) 880, interface (e.g., touchscreen and GUI) 890, power and control (e.g., circuits and logic) 900, control 910, communication (e.g., wireless transceiver) 915, and speaker 920.

The battery 850 provides low voltage direct current power to the mobile tank in order to perform processing, data acquisition, sensor, and actuator operations. The battery is connected to the battery charging circuit 860 for recharging purposes. The battery charging circuit 860 interacts with the battery charging circuit on the stationary filter to recharge the battery.

The processor 870 receives power from the battery and executes instructions from the control 910. The processor communicates digitally with the power and control 900, data acquisition circuits 880, touchscreen 890, and the battery charging circuit 860. The data acquisition circuits 880 convert analog information from the sensors in the system to digital signals for the processors.

The interface (e.g., touchscreen display) 890 is powered by the DC power. The display outputs graphics as instructed by the processor and control logic to generate the graphical user interface. The touchscreen 890 communicates physical touch commands from the user to the processor 870. The control 910 instructs the system based on the user commands and algorithms. In an example, the screen may be configured to display total volume of fluid used, total volume of drug used, desired concentration of drug, actual concentration of drug, desired flow rate, desired pressure in the tank, “Time Out” checklist for using the device, a review of drug delivery, and/or flow rates, etc.

In an example, the power and control 900 are powered by the battery 850. These circuits energize and control the position of the actuators. The control logic 910 may be implemented as a set of instructions that run on the processor. The wireless communication transceiver 915 sends and receives data between the mobile tank, stationary filter, and local area network. The user may indicate that the mobile tank may be returned to the stationary filter soon. The mobile tank then sends a wireless communication signal to the stationary filter to prepare for a dock. This may include activating the flow of water through the filters and beginning deionization in the CDI Subsystem. This may be an automated feature as well after the volume of the fluid in the tank falls below a threshold. The speaker 920 provides a method for user feedback. This may include audible feedback for warnings, button presses, alarms, selections, etc.

Logic may be implemented at least in part as computer software and/or firmware and/or hardwired. The mobile tank employs user selectable user profiles. The profiles may include information for a particular surgeon, nurse, or facility. The following information may be included in the profiles: pressure, temperature, and flow settings for a given end effector, drug delivery settings, end effector button functions, and/or warning volumes.

User profile information may be accessed and/or selected from another device, physical storage media, RFID reader, or wireless Ethernet device. The system may maintain usage data. This data may be stored on the stationary filter, mobile tank, on an email account, on a local device via Ethernet, a server location, remote data storage via cell phone connection, or an EMR database. Example data may include the following. Batch details, including time- and batch-stamped measurements for all sensors. Case details, including user profile selection, patient name, end effectors used, flow rates and pressures from the mobile tank, type of fluid used, and amount of fluid used. Drug infusion details, including name of drug, serial/lot number, concentration, total volume used, etc. System errors, faults, messages. Maintenance Status, including filter life, total volume of fluid purified, number of batches generated, etc.

The system may reorder concentrate as concentrate is used. The user may select the temperature of the fluid, up to about 40° C. This enables the user to select a fluid temperature best suited for the patient and the therapy being provided.

Other examples may include digital LCD display and membrane switches instead of Touchscreen and GUI. An AC mains plug and AC/DC power supply instead of or in addition to a rechargeable battery. An inverter for AC power of medical devices from the battery. A clear tank wall (made from a transparent or translucent polymer). LEDs emit light in to the tank to generate a colored glow depending on the status of the device, so that the user can determine the device status.

In another example, the graphical user interface 890 shows waste fluid tank volume through the volume and sensor DAQ and control 1120. The graphical user interface 890 shows a warning when the waste tank volume is approaching capacity, as detected by the volume and sensor DAQ and control 1120. An audible alarm sounds through the speaker 920 when the volume of the waste fluid tank is near or at capacity.

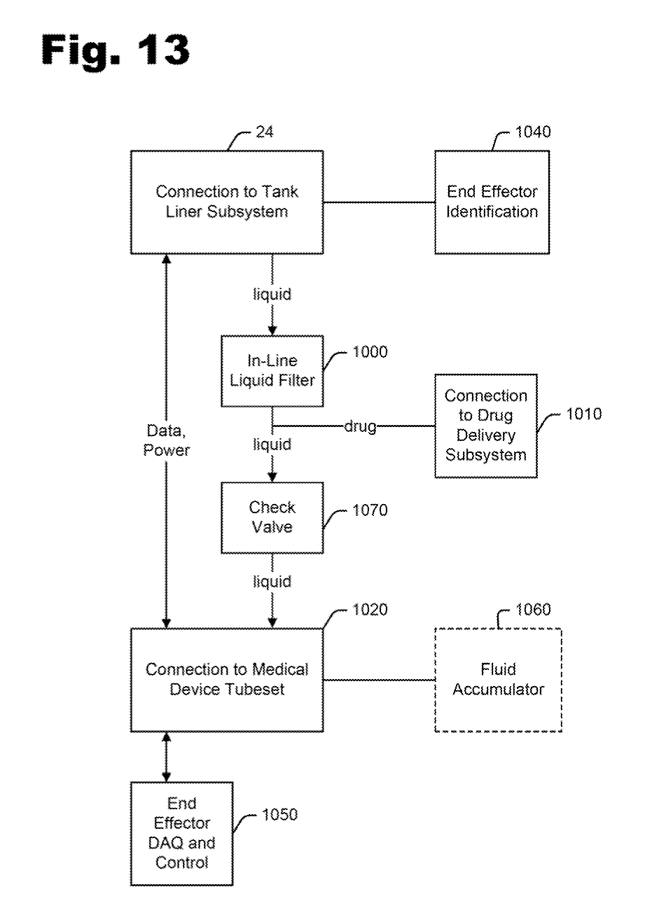

FIG. 13 illustrates an example end effector subsystem 32 of the example combined water purification and waste handling system. In an example, subsystem 32 is a single use consumable that carries fluid from the tank liner subsystem 24 to the device that consume the sterile fluid.

An example end effector subsystem includes in-line liquid filter 1000 (e.g., pore size less than or equal to about 0.22 microns, and flow rate greater than about 0.5 liters per minute at less than about 50 pounds per square inch), connection to drug delivery subsystem 26, connection to medical device tubeset 1020, connection to tank liner subsystem 24, end effector identification 1040, end effector DAQ and control 1050, fluid accumulator 1060, and check valve 1070.

Sterile fluid is received from the tank liner subsystem 24 from the connection to the end effector 700. The sterile fluid then enters the in-line liquid filter 1000, where bacteria, virus, and endotoxin may be removed from the fluid stream. This is a precautionary filter to remove any contaminant that existed on the connection surfaces or in the tank liner. The filter pore size may be less than or equal to 0.22 microns.

Upon exit from the in-line liquid filter 1000, the fluid joins the drug delivery connection 1010. Drug may be inserted in to the sterile fluid from the drug delivery subsystem 26. The liquid enters a check valve 1070 that prevents fluid from flowing back in to the in-line filter 1000 and tank liner subsystem 24.

The sterile fluid then enters a medical device through the connection to medical device tube set 1020. Examples of a compatible medical device may include an arthroscopy pump, laparoscopic suction-irrigator, pulsed lavage system, or a hemodialysis machine.

The end effector identification 1040 is located on the connection to the tank liner subsystem 24. This may be a bar code, RFID antenna, or any optical marking. This optical marking may communicate device type and operation parameters to the mobile tank.

The end effector DAQ and control 1050 accept and generate low voltage electrical and electro-mechanical inputs and outputs on the end effector.

A fluid accumulator 1060 for the sterile fluid is located in-line with the tubing to the medical device. In instances where high flow rates (greater than about 1.0 liter per minute) are used for short durations, the accumulator may provide a reservoir of ready fluid for the device. One example of the accumulator may be a standard intravenous (IV) fluid bag.

In other examples, a single use-consumable may be provided for generic connection to any device with bag spike. The consumable may include the connection to Tank Liner Subsystem, in-line liquid filter, connection to drug delivery subsystem 26, and common irrigation bag female connection. The filter with a positively charged filter membrane for endotoxin removal. Locate the connection to drug delivery subsystem 26 to the system before the in-line filter 1000. This enables sterilization of the drug concentrate.

In an example, the device does not use typical aseptic connections, but rather re-sterilizes after a non-aseptic connection. The addition of a filter to an irrigation device is a novel way of maintaining sterility of the fluid.

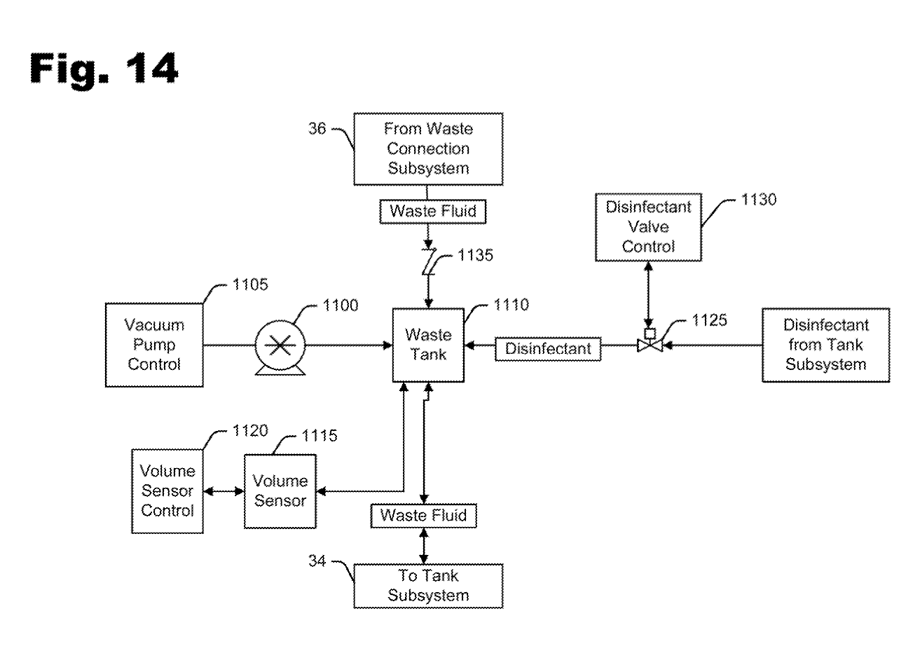

FIG. 14 illustrates an example waste tank subsystem 34 of the example combined water purification and waste handling system. In an example, subsystem 34 is a reusable waste fluid tank, and may include a vacuum pump 1100 (e.g., having a maximum vacuum pressure higher than about 100 mmHg), vacuum pump control 1105, waste tank 1110, volume sensor 1115 (e.g., having a range of about 0.1 to 100 liters and a resolution of about 0.1 liters), volume sensor control 1120, disinfectant valve 1125, disinfectant valve control 1130, and check valve 1135.

In an example, waste fluid enters the waste tank 1110 from the waste connection subsystem 36. The waste tank 1110 stores the waste fluid during the surgical or dialysis procedure. The vacuum pump 1100 draws a vacuum inside the waste tank 1110. The pump 1100 draws air in from the waste tank and has protection against drawing fluid in to the pump. The vacuum pump is driven by the vacuum pump control 1105. The volume sensor 1115 monitors the volume of fluid in the waste tank 1110. This sensor may be a load cell, pressure transducer, optical measurement technique, or ultrasonic measurement technique. The volume sensor control 1120 convert the transducer signal to a meaningful format and communicate the information to the processor. The disinfectant valve 1125 opens and closes based on the status of the system as dictated by the disinfectant valve control 1130. The waste tank 1110 is emptied by a connection to the Tank Subsystem when the mobile tank is connected to the stationary filter. The check valve 1135 prevents waste fluid from exiting the waste tank through the waste connection subsystem 36.

Logic may be configured such that when the mobile tank is connected to the stationary filter, the disinfectant valve 1125 opens and disinfectant is pumped in to the waste tank for cleaning purposes.

In other examples, the waste tank may be replaced by a disposable waste bag 817 with connections to the vacuum pump 1100, the waste connection subsystem 36, volume sensor 1115, and a tube for waste fluid evacuation, as described in the mobile tank mechanical subsystem 28.

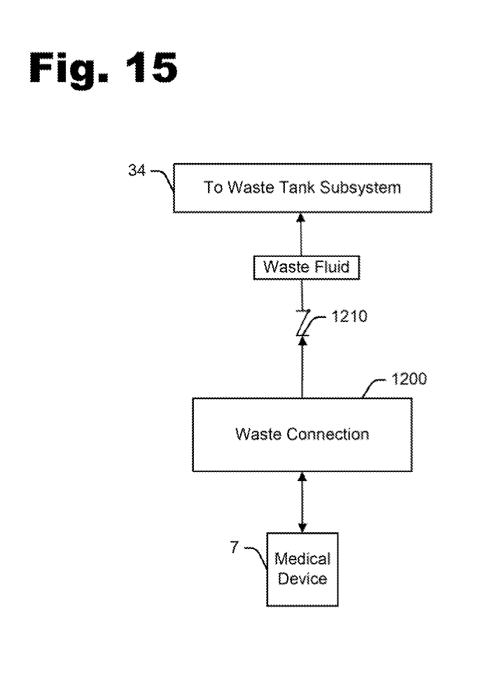

FIG. 15 illustrates an example waste connection subsystem 36 of the example combined water purification and waste handling system. In an example, subsystem 36 is the connection between the waste tank and a waste fluid-producing medical device 7. Components may include a check valve 1210, a waste connection 1200, and a medical device 7

In an example, a medical device 7 that generates waste fluids is connected to the waste connection 1200. This connection may be any medical tubing connection (e.g., luer lock) or a proprietary connection. The waste fluid is conducted through a check valve 1210 and to the waste tank subsystem 34. The waste connection subsystem 36 may be a disposable component or a permanent, capital component.

It is noted that the examples shown and described are provided for purposes of illustration and are not intended to be limiting. Still other examples are also contemplated.

1. A fluid handling system comprising:
a stationary filter having:
a Capacitive Deionization (CDI) subsystem,
a mix and filter subsystem, and
an output subsystem; and
a mobile tank having:
a tank subsystem, and
a tank liner subsystem; and
an end effector subsystem.

2. The fluid handling system of claim 1, wherein the mobile tank has a drug delivery subsystem.

3. The fluid handling system of claim 1, further comprising a pre-treatment subsystem.

4. The fluid handling system of claim 1, further comprising a filter station mechanical subsystem and a filter station electrical subsystem to support the subsystems of the stationary filter.

5. The fluid handling system of claim 1, further comprising a mobile tank mechanical subsystem and a mobile tank electrical subsystem to support the subsystems of the mobile tank.

6. The fluid handling system of claim 1, wherein at least one of the stationary filter, the mobile tank, and the end effector subsystem is configured as a boom-mounted system.

7. The fluid handling system of claim 1, therein the stationary filter, the mobile tank, and the end effector subsystem are configured as a transportable fluid management machine.

8. The fluid handling system of claim 1, wherein the stationary filter, the mobile tank, and the end effector subsystem are configured as a renal replacement therapy water machine to generate medical grade fluid for renal replacement treatment of a patient.

9. The fluid handling system of claim 1, wherein the stationary filter, the mobile tank, and the end effector subsystem are configured as a surgical water machine to generate medical grade fluid for a surgical procedure.

10. The fluid handling system of claim 1, further comprising an accumulator tank after the mix and filter subsystem and before the output subsystem, the accumulator tank configured to store fluid in advance of deployment to provide a batch of reserve fluid for fast deployment.

11. The fluid handling system of claim 1, further comprising a temperature control to maintain a desired temperature of fluid passing through a capacitive deionization chamber of the CDI subsystem.

12. The fluid handling system of claim 1, further comprising a waste handling system comprising a waste tank subsystem and a waste connection subsystem.

13. The fluid handling system of claim 12, wherein the waste handling system comprising a waste bag mass sensor and a warning mechanism to indicate waste bags are at or near capacity.

14. The fluid handling system of claim 1, further comprising at least one ultrafilter, the ultrafilter having a pore size rating of less than about 0.22 micron at fluid entrance to remove bacteria, virus, or endotoxin and maintain sterility of fluid after a non-aseptic connection.

15. The fluid handling system of claim 1, further comprising a total chlorine sensor.

16. A single-unit fluid handling system for purifying fluid for medical application and handling waste on the same device, comprising:
a stationary filter having:
a Capacitive Deionization (CDI) subsystem,
a mix and filter subsystem, and
an output subsystem; and
a mobile tank having:
a tank subsystem, and
a tank liner subsystem; and
an end effector subsystem; and
a waste handling component having a waste tank subsystem, and
wherein purified fluid is maintained in physical, chemical, and biological isolation from waste fluid on the same device.

17. The fluid handling system of claim 14, further comprising a drug delivery subsystem.

18. A fluid handling method to purify water for medical application, comprising:
providing a stationary filter having:
a Capacitive Deionization (CDI) subsystem to deionize influent fluid,
a mix and filter subsystem to combine concentrate with deionized water and then remove bacteria, virus, and endotoxin by ultrafiltration, and
an output subsystem to transfer fluid from the station filter; and
providing a mobile tank receiving fluid from the output subsystem, the mobile tank having:
a tank subsystem connected the mobile tank to the stationary filter, the tank subsystem inserting a tank liner into a fluid tank and pressurizing fluid in the fluid tank, and
a tank liner subsystem connecting output of the tank subsystem; and
providing an end effector subsystem to receive fluid from the output of the tank subsystem and deliver purified fluid to an end-use device.

19. The fluid handling method of claim 18, further comprising:
providing a pre-treatment subsystem in advance of the stationary filter, the pretreatment subsystem having an ultrafilter having a pore size rating of less than about 0.22 micron, the pretreatment subsystem pre-filtering influent fluid; and
providing a waste handling system to handle waste fluid, the waste handling system having a waste tank subsystem and a waste connection subsystem.

20. The fluid handling method of claim 18, further comprising drug delivery subsystem to inject a drug into purified fluid from the mobile tank.
